(19) United States
(12) Patent Application Publication
(10) Pub. No.: US 2010/0285558 A1
(43) Pub. Date: Nov. 11, 2010

(54) Apparatus and Method for High-Throughput Micro-Cell Culture with Mechanical Stimulation

(76) Inventors: Min-Hsien Wu, Kaohsiung City (TW); Wei-Chung Pan, Changhua County (TW); Chun-Yen Kuo, Pingtung County (TW)

Correspondence Address: WHYTE HIRSCHBOECK DUDEK S C;INTELLECTUAL PROPERTY DEPARTMENT, 555 EAST WELLS STREET, SUITE 1900, MILWAUKEE, WI, 53202, US

(21) Appl. No.: 12/437,803

(22) Filed: May 8, 2009

(51) Int. Cl. C12N 13/00; C12M 1/00

(52) U.S. Cl. 435/173.8; 435/305.1

(57) ABSTRACT

A method for high-throughput micro-cell culture with mechanical stimulation includes providing cells on a membrane, supplying a culture medium to the cells, and vibrating the membrane by exerting and varying a fluid pressure on the membrane such that the cells are mechanically stimulated through the membrane.

An apparatus for high-throughput micro-cell culture with mechanical stimulation includes a cell culture vessel and a fluid pressure supply unit. The cell culture vessel has the membrane, a culture medium chamber, and a pressure chamber. The fluid pressure supply unit is connected fluidly to the pressure chamber, and has a fluid pressure supply device and a control device. The membrane vibrates when the fluid pressure in the pressure chamber is varied. The apparatus provides mechanical stimulation to the cultured cells in a controllable manner.

### BACKGROUND OF THE INVENTION

1. Field of the Invention

This invention relates to a method and an apparatus for cell culture, more particularly to an apparatus and a method for high-throughput micro-cell culture with mechanical stimulation.

2. Description of the Related Art

Cell culture is a conventional technique that is widely used in biological research and can be generally classified into static cell culture and perfusion cell culture. Two equipments suitable for static cell culture are a multi-well microplate and a Petri dish. Compared to static cell culture, perfusion cell culture is able to provide a steady and quantifiable culture condition that is favorable for establishing a quantitative link between an extracellular stimulus and a cellular response. Consequently, perfusion cell culture has been broadly used in recent years.

However, a conventional device for perfusion cell culture has a large size, a complicated structure, and a high production cost, is unable to be easily operated, and is not appropriate for high throughput applications. The disadvantages of the device for perfusion cell culture may limit a range of application of the same.

Furthermore, in order to investigate a relationship between mechanical stimulation and cell physiology (or cell biochemistry), a new device that is suitable for perfusion cell culture, that can overcome the aforementioned disadvantages of the conventional device for perfusion cell culture, and that is capable of providing mechanical stimulation is required. For example, the new device can be operated to examine how mechanical stimulation may influence differentiation of stem cells or tissue growth regarding tissue engineering.

### SUMMARY OF THE INVENTION

Therefore, the object of the present invention is to provide an apparatus and a method for high-throughput micro-cell culture with mechanical stimulation in order to overcome the aforesaid drawbacks of the prior art.

According to one aspect of this invention, there is provided a method for high-throughput micro-cell culture with mechanical stimulation. The method comprises: providing cells on a membrane; supplying a culture medium to the cells; and vibrating the membrane by exerting and varying a fluid pressure on the membrane such that the cells are mechanically stimulated through the membrane.

According to another aspect of this invention, there is provided an apparatus for high-throughput micro-cell culture with mechanical stimulation. The apparatus includes a cell culture vessel and a fluid pressure supply unit.

The cell culture vessel has a membrane that is thereinside adapted to support cells, a culture medium chamber that is disposed on one side of the membrane, and a pressure chamber that is disposed on the other side of the membrane and that has a fluid pressure transmittable to the membrane.

The fluid pressure supply unit is connected fluidly to the pressure chamber, and has a fluid pressure supply device so as to supply a pressurized fluid to the pressure chamber, and a control device that is adapted to vary a pressure of the fluid supplied to the pressure chamber, thereby varying the fluid pressure. The membrane vibrates when the fluid pressure is varied.

### BRIEF DESCRIPTION OF THE DRAWINGS

Other features and advantages of the present invention will become apparent in the following detailed description of the preferred embodiments of this invention, with reference to the accompanying drawings, in which:

FIG. 1 is a schematic diagram of the preferred embodiment of an apparatus for high-throughput micro-cell culture with mechanical stimulation according to this invention;

FIG. 2 is an exploded perspective view to illustrate a cell culture vessel of the apparatus according to the preferred embodiment;

FIG. 3 is a sectional view to illustrate the cell culture vessel of the apparatus according to the preferred embodiment;

FIG. 4 is the same view as FIG. 3 but illustrating that a membrane of the cell culture vessel is elastically deformed by a fluid pressure; and

FIG. 5 is a flow chart to illustrate the preferred embodiment of a method for high-throughput micro-cell culture with mechanical stimulation according to this invention.

### DETAILED DESCRIPTION OF THE PREFERRED EMBODIMENTS

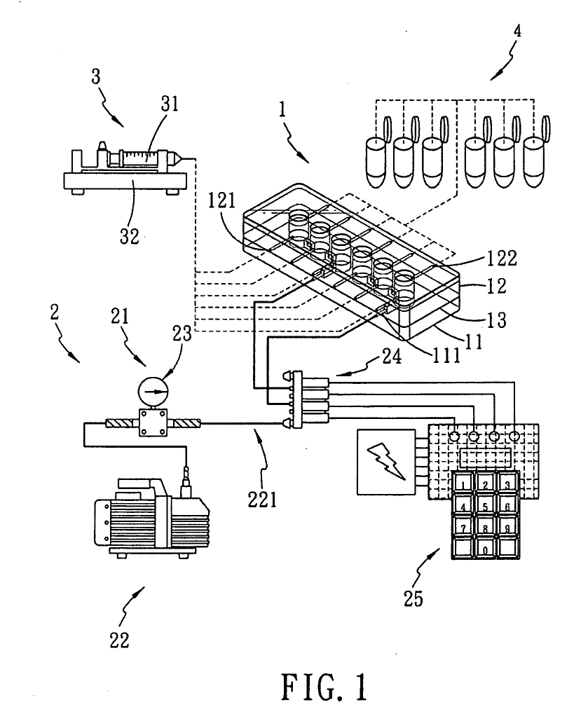

Referring to FIG. 1, according to the present invention, the preferred embodiment of an apparatus for high-throughput micro-cell culture with mechanical stimulation is a perfusion cell culture apparatus, and includes a cell culture vessel 1 and a fluid pressure supply unit 2.

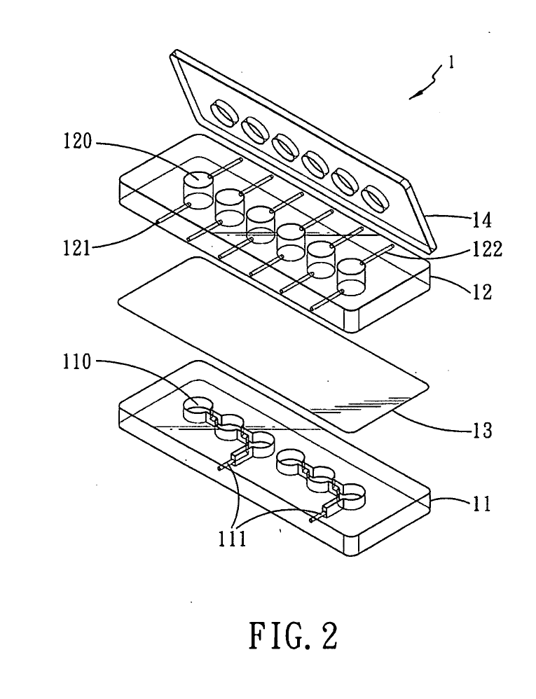

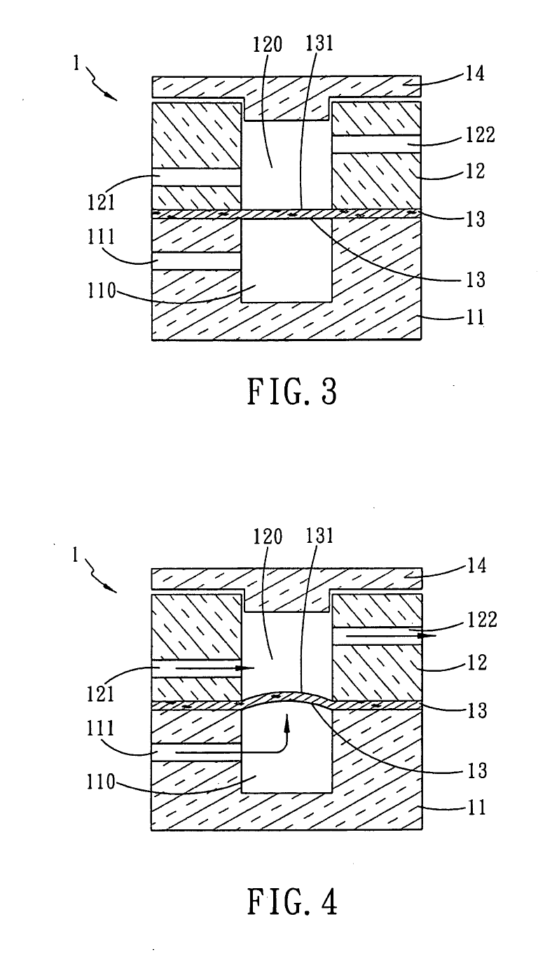

Referring to FIGS. 1, 2, and 3, the cell culture vessel 1 has a membrane 13 that is thereinside adapted to support a plurality of cells (not shown), six culture medium chambers 120 that are disposed on one side of the membrane 13, and six pressure chambers 110 that are disposed on the other side of the membrane 13 and that have a fluid pressure transmittable to the membrane 13. The three consecutive pressure chambers 110 on the right are in spatial communication, and the other three consecutive pressure chambers 110 on the left are in spatial communication. Each of the pressure chambers 110 corresponds in position to a respective one of the culture medium chambers 120.

Preferably, the cell culture vessel 1 is a multi-layered structure including a base layer 11 which has the pressure chambers 110, a top layer 12 which has the cell culture chambers 120, and a middle layer which forms the membrane 13, and which is disposed between the base layer 11 and the top layer 12 so that the membrane 13 is between the pressure chambers 110 and the culture medium chambers 120. The membrane 13 has a top surface 131 that faces the culture medium chambers 120 and that is adapted to attach the cells for 2-D cell culture or to place a construct for 3-D cell culture. In this embodiment, the cell culture vessel 1 further includes a cover 14 that is disposed on the top layer 12.

The base layer 11, the top layer 12, and the cover 14 are all made from polymer or plastics. The membrane 13 is made from an elastomer such as polydimethylsiloxane. The base layer 11, the top layer 12, and the cover 14 are formed by casting or other molding techniques such as injection molding or hot embossing. In the case of casting, a desired mold is first produced. Afterward, a predetermined amount of a polymer in liquid form is poured into the mold and allowed to solidify. Finally, the polymer that is in solid form and that has a desired shape is separated from the mold.

The membrane 13 is formed by a spin coating process or a molding process. A thickness of the membrane 13 is controlled by adjusting a rotation rate that is employed in the spin coating process. Subsequently, the membrane 13 is bonded to the base layer 11 and the top layer 12. A surface treatment (e.g., plasma oxidation) is conducted to improve the bonding of the membrane 13 to the base layer 11 and the top layer 12. The cover 14 is detachably connected to the top layer 12.

The fluid pressure supply unit 2 is connected fluidly to the pressure chambers 110, and has a fluid pressure supply device 22 so as to supply a pressurized fluid (not shown) to the pressure chambers 110 through a piping unit 221, and a control device 21 that is adapted to vary a pressure of the fluid supplied to the pressure chambers 110, thereby varying the fluid pressure. The membrane 13 vibrates when the fluid pressure is varied. In this embodiment, the fluid pressure supply device 22 is a pneumatic device that supplies a pressurized gas. It should be noted that the fluid pressure supply device 22 could be a hydraulic device that supplies a pressurized liquid in other embodiments.

The control device 21 of the fluid pressure supply unit 2 has a pressure/flow regulator 23 connected to the piping unit 221 so as to regulate flows of the fluid in the piping unit 221, and a set of electro-magnetic valves 24 connected to the piping unit 221 to control the pressurized fluid so that the pressurized fluid enters intermittently into the pressure chambers 110. The control device 21 further includes a control module 25 to control alternate opening and closing of the electromagnetic valves 24, and to control the pressure/flow regulator 23, thereby varying the fluid pressure in the pressure chambers 110. The frequency of vibration of the membrane 13 can therefore be controlled.

The apparatus further includes a culture medium supply device 3 that is connected fluidly to the cell culture chambers 120 of the top layer 12 and that is adapted to supply a culture medium (not shown) to the cell culture chambers 120, and a culture medium collecting device 4 that is connected fluidly to the cell culture chambers 120 and that is adapted to collect the culture medium from the cell culture chambers 120. In this embodiment, the culture medium supply device 3 includes a syringe 31 adapted to contain the culture medium, and a syringe pump 32 to actuate a plunger of the syringe 31 for expelling the culture medium out of the syringe 31. By means of the culture medium supply device 3, a predetermined amount of the culture medium can be supplied to the cells in the cell culture chambers 120 at predetermined intervals. It is noted that the culture medium supply device 3 can be a multi-syringe infusion pump or a peristaltic pump in other preferred embodiments.

The top layer 12 further has six inflow channels 121 (or more than six units for more high throughput applications) that are in fluid communication with the cell culture chambers 120 and the culture medium supply device 3, and six outflow channels 122 that are in fluid communication with the cell culture chambers 120 and the culture medium collecting device 4. The outflow channels 122 are higher than the inflow channels 121. Six inflow metal tubes (not shown) are respectively inserted into the six inflow channels 121 and are respectively connected to six inflow silica gel tubes (not shown) that are connected to the culture medium supply device 3. Similarly, six outflow metal tubes (not shown) are respectively inserted into the six outflow channels 122 and are respectively connected to six outflow silica gel tubes (not shown) that are connected to the culture medium collecting device 4.

Referring to FIGS. 1, 2, and 4, the fresh culture medium flows into the cell culture chambers 120 from the culture medium supply device 3 through the inflow channels 121 at a lower position. The waste culture medium flows to the culture medium collecting device 4 from the cell culture chambers 120 via the outflow channels 122 when the level of the culture medium rises to a higher level. Therefore, a constant environment is established for growth of the cells due to continuous supply of the fresh culture medium and continuous removal of the waste culture medium. A steady and quantifiable culture condition is achieved as well.

The base layer 11 further has two flow channels 111, each of which is in fluid communication with three of the pressure chambers 110 and the fluid pressure supply unit 2. The pressurized fluid is introduced into the pressure chambers 110 by virtue of the flow channels 111 such that the membrane 13 is pushed intermittently.

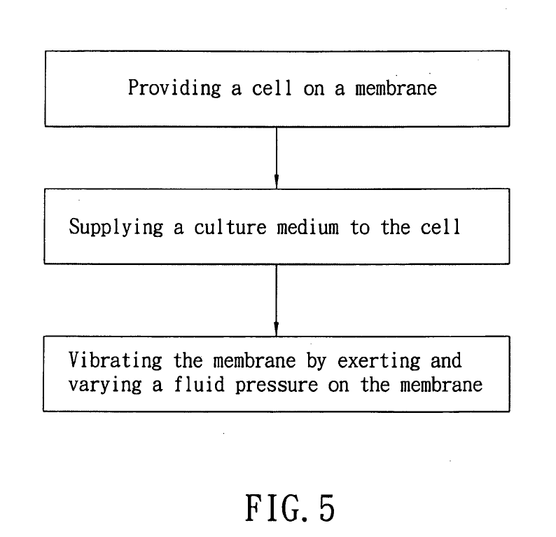

Referring to FIGS. 1 to 5, according to the present invention, the preferred embodiment of a method for high-throughput micro-cell culture with mechanical stimulation is performed by virtue of the aforementioned apparatus for high-throughput micro-cell culture with mechanical stimulation.

The method includes providing the cells on the membrane 13, supplying the culture medium to the cells, and vibrating the membrane 13 by exerting and varying the fluid pressure on the membrane 13 such that the cells are stimulated through the membrane 13.

Preferably, the method further includes sterilizing the cell culture vessel 1 before providing the cells on the membrane 13 of the cell culture vessel 1. Alcohol, a retort (such as an autoclave), UV light, a suitable gas, or other sterilization means can be utilized to sterilize the cell culture vessel 1.

The cells may be attached to the top surface 131 of the membrane 13 or be entrapped in a 3-D construct placed on the top surface 131 of the membrane 13 after sterilizing the cell culture vessel 1. Examples of the cells are articular cartilage cells or other cells appropriate for mechanical stimulation.

In operation, the culture medium is supplied to the cells by allowing the culture medium to flow into the cell culture chambers 120, and to flow over the cells (for 2-D cell culture) or to flow through the 3-D culture construct (for 3-D cell culture). Once the fluid pressure supply unit 2 is operated, the pressurized fluid is injected into the pressure chambers 110 such that the fluid pressure in the pressure chambers 110 is increased.

Referring to FIG. 4, the membrane 13 is elastically deformed and moved towards the cell culture chambers 120 when the fluid pressure in the pressure chambers 110 is increased. The membrane 13 restores to its original shape that is flat when the fluid pressure in the pressure chambers 110 is decreased by releasing the fluid pressure to atmosphere through the control of the electro-magnetic valves 24. By alternately increasing and decreasing the fluid pressure, the membrane 13 is vibrated. The cells on the membrane 13 are hence mechanically stimulated.

The cell culture vessel 1 is able to be miniaturized, is suitable for high throughput applications, can be easily operated with the fluid pressure supply unit 2 to provide mechanical stimulation, and has a simple structure and a low production cost. Consequently, the cell culture vessel 1 is suitable to be further developed into a disposable cell culture device.

While the present invention has been described in connection with what are considered the most practical and preferred embodiments, it is understood that this invention is not limited to the disclosed embodiments but is intended to cover various arrangements included within the spirit and scope of the broadest interpretation and equivalent arrangements.

What is claimed is:

1. A method for high-throughput micro-cell culture with mechanical stimulation, comprising:
providing cells on a membrane;
supplying a culture medium to the cells; and
vibrating the membrane by exerting and varying a fluid pressure on the membrane such that the cells are stimulated through the membrane.

2. The method of claim 1, wherein the culture medium is supplied to the cells by allowing the culture medium to flow over and through the cells.

3. The method of claim 1, wherein the fluid pressure is varied by alternately increasing and decreasing the fluid pressure.

4. The method of claim 1, wherein the fluid pressure is a pneumatic pressure.

5. The method of claim 1, wherein the fluid pressure is a hydraulic pressure.

6. The method of claim 1, wherein the membrane is made from an elastomer.

7. An apparatus for high-throughput micro-cell culture with mechanical stimulation, comprising:
a cell culture vessel having a membrane that is thereinside adapted to support cells, a culture medium chamber that is disposed on one side of said membrane, and a pressure chamber that is disposed on the other side of said membrane and that has a fluid pressure transmittable to said membrane; and
a fluid pressure supply unit connected fluidly to said pressure chamber, and having a fluid pressure supply device so as to supply a pressurized fluid to said pressure chamber, and a control device that is adapted to vary a pressure of the fluid supplied to said pressure chamber, thereby varying the fluid pressure, said membrane vibrating when the fluid pressure is varied.

8. The apparatus as claimed in claim 7, wherein said cell culture vessel is a multi-layered structure that includes a base layer having said pressure chamber, a top layer having said cell culture chamber, and a middle layer forming said membrane and disposed between said base layer and said top layer.

9. The apparatus as claimed in claim 8, wherein said middle layer of said cell culture vessel is made from an elastomer, said middle layer being bonded to said base and top layers of said cell culture vessel.

10. The apparatus as claimed in claim 8, wherein said middle layer is formed by a spin coating process or a molding process.

11. The apparatus as claimed in claim 8, further comprising a culture medium supply device connected fluidly to said cell culture chamber of said top layer and adapted to supply a culture medium to said cell culture chamber, and a culture medium collecting device connected fluidly to said cell culture chamber and adapted to collect the culture medium from said cell culture chamber.

12. The apparatus as claimed in claim 10, wherein said top layer further has an inflow channel that is in fluid communication with said cell culture chamber and said culture medium supply device, and an outflow channel that is in fluid communication with said cell culture chamber and said culture medium collecting device.

13. The apparatus as claimed in claim 8, wherein said base layer further has a flow channel that is in fluid communication with said pressure chamber and said fluid pressure supply unit.

14. The apparatus as claimed in claim 7, wherein said fluid pressure supply device is a pneumatic device.

15. The apparatus as claimed in claim 7, wherein said fluid pressure supply device is a hydraulic device.

16. The apparatus as claimed in claim 7, wherein said control device has an electromagnetic valve.
